(19) United States
(12) Patent Application Publication
(10) Pub. No.: US 2010/0062492 A1
(43) Pub. Date: Mar. 11, 2010

(54) Method For Preparing Icariside II, Cosmetic Composition Containing The Same and the Use Thereof For Skin Whitening

(75) Inventors: Jun Seong Park, Suwon-si (KR); Hye Yoon Park, Annyang-si (KR); Ho Sik Rho, Yongin-si (KR); Soo Mi Ahn, Suwon-si (KR); Duck Hee Kim, Seoul (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 12/440,984

(22) PCT Filed: Sep. 19, 2007
(86) PCT No.: PCT/KR07/04557

§ 371 Date: Apr. 17, 2009

(30) Foreign Application Priority Data
Sep. 19, 2006 (KR) 10-2006-0090795

(51) Int. Cl. C07H 17/065; C12P 19/60

(52) U.S. Cl. 435/75; 536/8

(57) ABSTRACT

Disclosed are a method for preparing icariside II represented by Formula 1, which inhibits the glycosylation of glycoprotein enzyme tyrosinase by inhibiting the enzymatic activity of alpha-glucosidase, which is an enzyme in the glycosylation of tyrosinase, as well as a skin whitening composition.

### TECHNICAL FIELD

The present invention relates to a method for preparing icariside II, a cosmetic composition containing the same, and the use of the composition for skin whitening, and more particularly to a method for preparing icariside II represented by Formula 1, which inhibits the glycosylation of glycoprotein enzyme tyrosinase by inhibiting the enzymatic activity of alpha-glucosidase, which is an important enzyme in the glycosylation of tyrosinase, as well as a cosmetic composition and the use thereof for skin whitening:

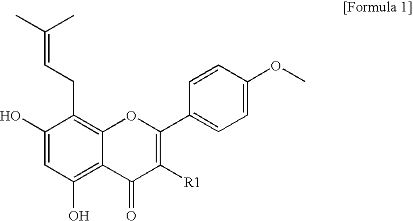

wherein R1 is rhamnopyranose.

### BACKGROUND ART

Various factors are involved in determining human skin color, and among them, factors, such as the activity of melanocytes, which make melanin pigments, the distribution of blood vessels, the thickness of the skin, and the presence or absence of pigments (e.g., carotenoid, bilirubin, etc.) in the human body, are of importance.

The most important factor among them is black pigment melanin, which is produced by the action of various enzymes such as tyrosinase, in human melanocytes. The formation of the melanin pigment is influenced by genetic factors, hormone secretion, physiological factors associated with stresses, and environmental factors such as UV light irradiation.

The melanin pigment, which is produced in melanin cells on the body skin, is a phenolic polymer, having a complex of a black pigment and a protein. It blocks the sun's ultraviolet rays to protect the skin organs under the dermis and, at the same time, removes free radicals generated in skin tissues to protect proteins and genes in the skin.

However, melanin, produced by internal or external stress stimuli in the skin, is a stable substance, which is not removed even when the stresses disappear, until it is discharged by skin keratinization. Thus, when melanin is produced in an unnecessarily large amount, hyperpigmentations, such as discoloration, freckles and spots, which are unfavorable in terms of beauty, will occur.

As people who like outdoor activity have increased with an increase in leisure population, the need to prevent melanin pigmentation caused by UV light has increased.

In order to satisfy this need, ascorbic acid, kojic acid, albutin, hydroquinone, glutathione, or derivatives thereof, or substances having tyrosinase inhibitory activity, have been used in cosmetics or medical drugs. However, the use thereof has been limited due to insufficient whitening effects and various problems, such as skin safety, and formulation and stability, which occur when they are added to cosmetics.

Tyrosinase, a melanin biosynthesis enzyme, is a glycoprotein, which is produced in vivo through a glycosylation process. When a problem occurs in the glycosylation process to cause abnormality in the glycose moiety of tyrosinase, tyrosinase will not be transferred to melanosome for intracellular melanin biosynthesis or will not exhibit tyrosinase activity, even if it is transferred to melanosome, and thus melanin cannot be produced (The Journal of Investigated Dermatology, 83, 196-201, 1984, The Journal of Biological Chemistry, 272(25), 15796-15803, 1997). Many enzymes are involved in the glycosylation process, and among them, alpha-glucosidase is an important enzyme (The Journal of Biological Chemistry, 272(25), 15796-15803, 1997). If the enzymatic activity of alpha-glucosidase can be inhibited, the glycosylation of tyrosinase can be inhibited, resulting in a skin whitening effect.

### DISCLOSURE

### Technical Problem

Accordingly, the present inventors have examined the alpha-glucosidase inhibitory activities of microorganisms from various natural substances in studies, focused on solving the above-described problems and finding a raw material having a more excellent whitening effect. As a result, the present inventors have found that icariside II, which is the flavonoid component of Epimedium plant extract, shows an excellent effect of inhibiting alpha-glucosidase enzyme activity, and thus has an excellent effect as a skin whitening agent, thereby completing the present invention.

Therefore, it is an object of the present invention to provide a method for preparing icariside II represented by Formula 1, and a skin whitening cosmetic composition containing the same as an active ingredient.

### Technical Solution

To achieve the above object, the present invention provides a method for preparing icariside II represented by Formula 1, a skin whitening cosmetic composition containing the same as an active ingredient, and the use thereof for skin whitening:

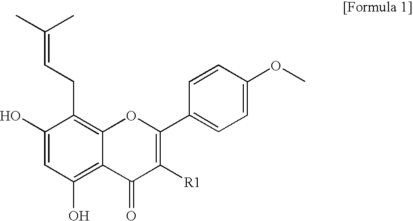

wherein R1 is rhamnopyranose.

Hereinafter, a process for preparing icariside II according to the present invention will be described in further detail.

Icariside II, which is contained in the cosmetic composition according to the present invention, can be prepared according to the following two methods.

First, icariside II can be prepared by purifying it directly from a plant containing icatiside II.

The plant containing icariside II according to the present invention is preferably Epimedium-derived plant extract, and specific examples of the plant extract include, but are not limited to, extracts of Epimedium brevicornum Maxim., Epimedium grandiflorum Morr., Epimedium koreanum Nakai, Epimedium pubescens Maxim., Epimedium sagittatum Maxim. and Epimedium wushanense.

Also, in the present invention, at least one organic solvent selected from the group consisting of ethanol, methanol, butanol, ether, ethylacetate, chloroform, and mixtures thereof with water, may be used. Preferably, 80% ethanol may be used.

In the present invention, the method of obtaining icariside II from a plant using water or an organic solvent is as follows. That is, a plant is added to about 1-6-fold volume (preferably about 3-fold volume) of water, or at least one organic solvent selected from the group consisting of ethanol, methanol, butanol, ether, ethyl acetate and chloroform, or a mixed solvent of the organic solvent(s) with water, in which the volume of the organic solvent is 10-50% (v/v). The plant in the solvent is defatted by extracting it 1-5 times with stirring at room temperature, and the defatted plant is added to about 1-8-fold volume (preferably about 4-fold volume) of water or an organic solvent and extracted 1-5 times under reflux. Then, the extracted plant is settled at 10-20° C. for 1-3 days.

The settled material is filtered and centrifuged into residue and a filtrate, and the filtrate is concentrated under reduced pressure. The resulting extract is suspended in water and depigmented with, for example, ether. Then, the aqueous layer is extracted 1-5 times with, for example, butanol, and the resulting organic solvent layer is concentrated under reduced pressure. The resulting extract is dissolved in a small amount of methanol or the like, and a large amount of ethyl acetate or the like is added thereto. The formed precipitate is dried, thus obtaining an extract containing icariside II. The extract is subjected to silica gel column chromatography (chloroform:methanol=8:1-4:1), thus obtaining icariside II.

Second, icariside II can be prepared by obtaining an icariin-containing plant extract and removing a glucose moiety from icariin in the plant extract. Icariin or a plant extract containing the same can be obtained in the same manner as the above-described method of obtaining icariside II, and the method of removing the glucose moiety of icariin can be performed either using an enzyme capable of selectively removing glucose without acting on rhamnose, or using a microorganism producing said enzyme.

In the present invention, the enzyme or the microorganism producing the enzyme may be an enzyme degrading the glucose linkage or a microorganism producing the enzyme. The enzyme allows icariside II to be prepared by selectively removing a glucose moiety from icariin without degrading rhamnose.

As the enzyme, one or more selected from the group consisting of amylase, glucosidase, arabinosidase, xylosidase, cellulase, glucuronidase, galactosidase and amyloglucosidase may be used.

Also, as the microorganism producing the enzyme may be one or more selected from the group consisting of Aspergillus sp., Bacillus sp., Penicillium sp., Rhizopus sp., Rhizomucor sp., Talaromyces sp., Bifidobacterium sp., Mortierella sp., Cryptococcus sp. and Microbacterium sp.

In the case where the enzyme is used, icariin or a plant extract containing the same is dissolved in a 5-20-fold volume (preferably about 20-fold volume) of acidic buffer solution, and the enzyme is then added thereto. The solution is stirred at about 37° C. for about 40-55 hours, and preferably about 48 hours, while the elimination rate of the substrate is examined by thin layer chromatography. When the substrate is completely eliminated, the reaction solution is heated in hot water (80-100° C.) for 5-15 minutes to terminate the hydrolysis reaction and is collected.

In the case where the microorganism producing the enzyme is used, icariin or a plant extract containing the same is dissolved in a 5-10-fold volume (preferably about 10-fold volume) of ionic water, and the solution is sterilized at about 121° C. for 30 minutes and then cooled to about 30° C. Then, a pre-cultured microorganism is inoculated into the solution in an amount of 5-10% based on the solution and cultured at 30° C. for 2-5 days, and preferably for 5 days. Then, the elimination rate of the substrate is examined by thin layer chromatography, and when the substrate is completely eliminated, the hydrolysis reaction is terminated. The culture medium is centrifuged at 5,000-10,000 rpm, and the precipitate is washed three times with distilled water and centrifuged again, and the precipitate is collected as the reaction product.

As described above, hydrolysis is carried out using the enzyme or the microorganism producing the enzyme, and the resulting reaction solution is concentrated under reduced pressure to remove the solvent. The residue is mixed with alcohol and stirred 1-5 times, and the precipitated salts are removed by filtration. The filtrate is concentrated under reduced pressure, thus obtaining a crude product. The crude product is subjected to silica gel column chromatography (chloroform:methanol=8:1-4:1), thus obtaining icariside II.

Icariside II prepared according to the present invention has an excellent effect of inhibiting alpha-glucosidase activity, acting on tyrosinase glycosylation, and thus shows an excellent skin whitening effect of inhibiting the production of melanin.

In another aspect, the present invention provides a cosmetic composition containing said icariside II as an active ingredient, and the use thereof for skin whitening.

There is no particular limitation on the formulation of the cosmetic composition according to the present invention. For example, the inventive composition can be formulated into skin lotion, milk lotion, massage cream, nourishing cream, packs, gel, or skin adhesive type cosmetic products. The content of said icariside II in the composition may be 0.0001-10 wt % based on the total weight of the composition.

Also, in the cosmetic compositions having the respective formulations, components other than said icariside II can be suitably selected without difficulty by one skilled in the art depending on the formulation or intended use of the composition.

### ADVANTAGEOUS EFFECTS

As described above, it was found that the cosmetic composition containing icariside II, separated from Epimedium plant extract or produced from icariin using the enzyme or the microorganism producing the enzyme, inhibited the activity of alpha-clucosidase to interfere with the normal glycosylation of tyrosinase, thus showing a skin whitening effect due to the effect of improving pigmentation caused by ultraviolet rays (UV). Accordingly, the composition containing icariside II according to the present invention will be useful as a skin whitening cosmetic composition or pharmaceutical composition.

### DESCRIPTION OF DRAWINGS

FIG. 1 depicts electrophoresis photographs of a control group (a), deoxynojirimycin (b), icariin (c) and icariside II (d).

### MODE FOR INVENTION

Hereinafter, the present invention will be described in further detail with reference to examples. It is to be understood, however, that these examples are illustrative only, and the scope of the present invention is not limited thereto.

### Example 1

### Preparation of Icariside II by Extraction

2 kg of the dried leaf of Epimedium koreanum Nakai was added to 6 liters of hexane and extracted three times with stirring at room temperature. 1 kg of the defatted plant leaf was added to 4 liters of methanol, extracted three times under reflux, and then settled at 15° C. for 1 day. Then, the settled material was filtered through filter cloth and centrifuged into residue and a filtrate. The filtrate was concentrated under reduced pressure. The resulting extract was suspended in water and extracted 5 times with ether to remove pigments, and the aqueous layer was extracted one time with 500 ml of 1-butanol. The resulting total 1-butalon layer was concentrated under reduced pressure to obtain a 1-butanol extract, which was then dissolved in a small amount of methanol. The solution was added to a large amount of ethyl acetate, and the formed precipitate was dried, thus obtaining an extract containing icariside II. The obtained extract was purified by silica gel column chromatography (filled with 500 g of silica gel). Herein, chloroform and methanol were used as developing solvents, fractions were collected at a concentration gradient of chloroform:methanol of 10:1-2:1, and 1.5 g of icariside II was collected from these fractions.

### Example 2

### Preparation of Icariside II Using Cellulose

10 g of icariin was dissolved in 500 ml of 0.1 M acetate buffer solution (pH 4.5), and 0.5 g of cellulase (Sigma) was added thereto. The solution was stirred in a water bath at 37° C. for 48 hours, while it was periodically examined by thin layer chromatography. When icariin was completely eliminated, the reaction solution was heated in hot water (80-100° C.) for 10 minutes to terminate the reaction, and then concentrated under reduced pressure to remove the solvent. The residue was added to 200 ml of ethanol, and the solution was stirred three times and filtered to remove the precipitate, and the filtrate was concentrated under reduced pressure, thus obtaining a crude product. The crude product was separated by silica gel column chromatography (chloroform:methanol=8:1-4:1), thus obtaining 7.5 g of icariside II.

### Example 3

### Preparation of Icariside II Using Beta-Glucosidase

10 g of icariin was dissolved in 500 ml of 0.1 M acetate buffer solution (pH 5.5), and 0.5 g of beta-glucosidase (Sigma) was added thereto. The solution was stirred in a water bath at 25° C. for 48 hours, while it was periodically examined by thin layer chromatography. When icariin was completely eliminated, the reaction solution was heated in hot water (80-100° C.) for 10 minutes to terminate the solution, and then concentrated under reduced pressure to remove the solvent. The residue was added to 200 ml of ethanol, and the solution was stirred three times and filtered to remove the precipitate. The filtrate was concentrated under reduced pressure, thus obtaining a crude product. The crude product was separated by silica gel column chromatography (chloroform:methanol=8:1-4:1), thus obtaining 6.9 g of icariside.

### Example 4

### Preparation of Icariside II Using Amylase

10 g of icariin was dissolved in 500 ml of 0.1 M acetate buffer solution (pH 5.5), and 0.5 g of amylase (Sigma) was added thereto. The solution was stirred in a water bath at 25° C. for 48 hours, while it was periodically examined by thin layer chromatography. When icariin was completely eliminated, the reaction solution was heated in hot water (80-100° C.) for 10 minutes to terminate the solution, and then concentrated under reduced pressure to remove the solvent. The residue was added to 200 ml of ethanol, and the solution was stirred three times and filtered to remove the precipitate. The filtrate was concentrated under reduced pressure, thus obtaining a crude product. The crude product was separated by silica gel column chromatography (chloroform:methanol=8:1-4:1), thus obtaining 7.3 g of icariside.

### Example 5

### Preparation of Icariside II Using Aspergillus niger

10 g of icariin was dissolved in 100 ml of ionic water, sterilized at 121° C. for 30 minutes and cooled to 30° C. Then, pre-cultured Aspergillus niger KCCM 11885 was inoculated into the icariin solution in an amount of 5-10% based on the solution and cultured at 30° C. for 5 days. Then, the elimination rate of icariin was examined by thin layer chromatography, and when icariin was completely eliminated, the reaction was terminated. The culture medium was centrifuged at 5,000-10,000 rpm, and the collected precipitate was washed three times with distilled water and centrifuged, thus collecting the reaction solution as the precipitate. The precipitate was added to 200 ml of ethanol, and the solution was stirred three times and filtered to remove the precipitate. The filtrate was concentrated under reduced pressure, thus obtaining a crude product. The crude product was separated by silica gel column chromatography (chloroform:methanol=8:1-4:1), thus obtaining 6.5 g of icariside.

### Test Example 1

### Identification of Icariside II

The products obtained in Examples 1-5 were identified (Varian Gemini 2000 300 MHz, Varian) and, as a result, they showed the following characteristics.

<Physical and chemical characteristics of icariside II>

Characteristic: light yellow-colored microcrystal

Positive FAB-MS: 515 [M+H]

¹H NMR: (DMSO-d6) δ: 0.79 (3H, d, 6, Me-5″), 1.63 & 1.68 (6H, br s, Me-11), 3.03 (1H, qd, 6, 9.5, H5″), 3.14 (1H, dd, 9, 9.5, H4″), ca 3.4 (2H-9, overlapping with the signals of H2O), 3.47 (1H, br, H3″), 3.85 (3H, s, OMe-4′), 3.98 (1H, br, H2″), 5.15 (1H, br t, 7, H10), 5.26 (1H, d, 1.5, H1″), 6.31 (1H, s, H6), 7.12 (2H, d, 9, H3′, 5′), 7.86 (2H, d, 9, H2′, 6′), 12.52 (1H, s, OH-5)

¹³C-NMR: (DMSO-d6) δ: 156.2, 133.8, 177.1, 103.6, 158.1, 97.8, 160.9, 105.4, 153.8, 21.0, 121.7, 130.3, 17.6, 25.2, 121.8, 129.7, 113.5, 160.5, 55.2, 101.4, 69.7, 70.0, 70.2, 70.8, 17.3.

Acid hydrolysates: icaritin and rhamnose

### Test Example 2

### Test of Effect of Icariside II

1. Test of Alpha-Glucosidase Inhibitory Effect

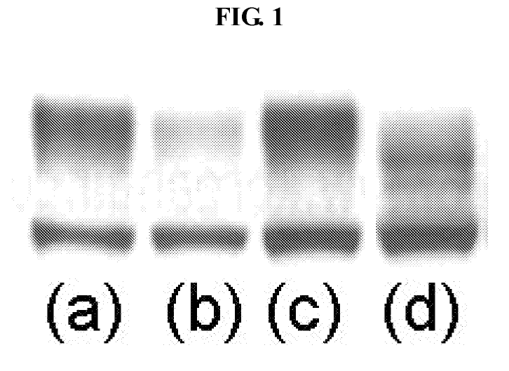

50 U/ml of alpha-glucosidase (Sigma) was added to each of 0.01 ml of icariin, contained in 1 ml of a coenzyme solution in an amount of 10 mg/ml, icariside II prepared in Examples 1-5, and deoxynojirimycin, and then left to stand for 5 minutes. In this regard, the deoxynojirimycin was used as a positive control group. Each of the samples was measured for absorbance at 405 nm to determine initial absorbance, and 0.05 ml of 5 mM p-nitrophenyl-α-D-glucopyranoside as a substrate was added thereto and subjected to an enzymatic reaction at 37° C. for 5 minutes. Then, each sample was measured for absorbance at 405 nm, and the enzyme activity inhibition thereof was calculated according to Math Figure 1.

Alpha-glucosidase activity inhibition (%)=100−(absorbance of each test sample/absorbance of control group×100) [Math Figure 1]

TABLE 1

| | Activity |
|---|---|
| Test samples | inhibition (%) |
| Untreated group | 100.0 |
| Icariin | 98.8 |
| Example 1 | 38.2 |
| Example 2 | 38.8 |
| Example 3 | 37.5 |
| Example 4 | 37.9 |
| Example 5 | 37.8 |
| Deoxynojirimycin | 41.1 |

As can be seen in Table 1 above, icariside II, prepared in Examples 1-5 of the present invention, had an alpha-glucosidase activity inhibitory effect similar to that of deoxynojirimycin.

2. Effect on Glycosylation of Human Melanoma Cell Tyrosinase

In order to examine the effect of icariside II on the glycosylation of tyrosinase in human melanoma cells, the following test was carried out. First, human melanoma cell HM3KO cells (Y. Funasaka, Department of dermatology, Kobe university school of medicine, 5-1 Kusunoki-cho 7-chrome, Chuo-ku, Kobe 650, Japan) were cultured in a minimum essential medium (MEM), containing 10% bovine fetal serum, under conditions of 37° C. and 5% CO₂. The cultured cells were placed in 75 cm² flasks at a cell density of 3×10⁵ cells and left to stand overnight, such that the cells adhered to the flask wall. From the next day, the medium was replaced with a fresh medium, containing each of icariin icariside II of Example 1 and deoxynojirimycin in an amount of 0.05%. Herein, the deoxynojirimycin was used as a positive control group. The medium was replaced with a fresh medium containing each of the samples at a 1-2-day interval, and the cells were cultured to confluency in the flasks. When the cells reached confluency, the cells were collected, added to lysis buffer (2% CHAPS in 50 mM Hepes and 200 mM NaCl, pH 7.5, protease inhibitors) and ultrasonically disrupted. The disrupted cell solution was centrifuged at 4° C. and 12000 rpm for 10 minutes, unbroken cells and melanin were separated and removed, and only the supernatant was collected. Endoglycosidase H (125 units) was added to the supernatant (protein amount: 20 g), and the supernatant was subjected to an enzymatic reaction at 37° C. for 1 hour. Then, proteins in the supernatant were separated according to size by electrophoresis. Among the proteins separated by electrophoresis, tyrosinase could be observed by an immune reaction with an antibody. That is, tyrosinase, in which sugar residue was normally formed, appeared as an about 72-kD glycoprotein, because the sugar residue was not enzymatically degraded by endoglycosidase H, whereas tyrosinase, in which normal glucose residue was not formed due to the inhibition of enzymatic activity of alpha-glucosidase involved in the glycosylation process, appeared as an about 60-kD protein, because the glucose residue was hydrolyzed by endoglycosidase H.

Referring to FIG. 1, it can be observed that tyrosinase, treated with a control group (a) as a medium containing no sample, or with icariin (c), showed large size (about 72 kD), because the glucose residue was not degraded by endoglycosidase, whereas melanocyte tyrosinase, treated with deoxynojirimycin (b) or icariside II (d), showed small size (about 60 kD), because the glucose residue was completely degraded by endoglycosidase. Such results indicate that icariside II can inhibit the formation of glycoprotein of tyrosinase, thus inhibiting the enzymatic activity of tyrosinase.

3. Test of Whitening Effect on Human Skin

In order to examine the whitening effect of icariside II on human skin, the following test was carried out.

First, a perforated opaque tape having a diameter of 1.5 cm was attached to the upper arm of each of 12 healthy men. Then, UV rays (UVB) were irradiated to each of the subjects at a dose 1.5-2 times higher than the minimal erythema dose of each subject, thus inducing skin's blackness.

After the UV irradiation, each of a 1% solution of icariside (1,3-butyleneglycol:ethanol=7:3, as a vehicle), prepared in Example 1, a 1% solution of hydroquinone, a 1% solution of vehicle (negative control group), was applied to the skin of each subject. A change in the state of the subject's skin was observed for 10 weeks. The skin color was measured with a colorimeter (Minolta, Japan) at a 1-week interval.

Then, the difference (ΔL*) in skin color between the time point of application and the time point of completion of application of each sample was calculated according to Math Figure 2, and the calculation results are shown in Table 2 below. Meanwhile, the whitening effect of each sample is evaluated by comparing ΔL* between the site applied with each sample and a control site not applied with each sample, in which a ΔL* value of about indicates that the pigmentation was clearly improved, and a ΔL* value higher than about 1.5 indicates that the sample has a whitening effect.

ΔL*=L* value at time point of completion of application−L* value at time point of start of application [Math Figure 2]

TABLE 2

| | Lightless (ΔL*) |
|---|---|
| Test samples | of skin color |
| Icariside II of Example 1 | 1.97 ± 0.25 |
| Hydroquinone (positive control group) | 1.90 ± 0.11 |
| Vehicle (negative control group) | 0.50 ± 0.15 |

As can be seen in Table 2 below, icariside prepared in Example 1 of the present invention showed skin color lightness similar to that of hydroquinone.

Hereinafter, the formulation of the inventive composition will be described with reference to formulation examples. It is to be understood, however, that these formulation examples are illustrative only, and the scope of the present invention is not limited only thereto.

Formulation Example 1: Preparation of soap

| Components | Content (wt %) |
|---|---|
| Icariside II | 1.00 |
| Oils and fats | qs |
| Sodium hydroxide | qs |
| Sodium chloride | qs |
| Perfume | small amount |

Purified water was added to the above components to make a total of 100 wt %, and soap having said composition was prepared.

Formulation Example 2: Preparation of lotion

| | Content |
|---|---|
| Components | (wt %) |
| Icariside II | 3.00 |
| L-ascorbic acid-2-magnesium | 1.00 |
| phosphate | |
| Water soluble collagen | 1.00 |
| (1% aqueous solution) | |
| Sodium citrate | 0.10 |
| Citric acid | 0.05 |
| Licorice extract | 0.20 |
| 1,3-butylene glycol | 3.00 |

Purified water was added to the above components to make a total of 100 wt %, and lotion having said composition was prepared.

Formulation Example 3: Preparation of cream

| | Content |
|---|---|
| Components | (wt %) |
| Icariside II | 1.00 |
| Polyethylene glycol | 2.00 |
| monostearate | |
| Self-emulsifying glycerin | 5.00 |
| monostearate | |
| Cetyl alcohol | 4.00 |
| Squalene | 6.00 |
| Glycerol | 6.00 |
| Sphingoglycolipid | 1.00 |
| 1.3-butylene glycol | 7.00 |

Purified water was added to the above components to make a total of 100 wt %, and cream having said composition was prepared.

Formulation Example 4: Preparation of pack

| | Content |
|---|---|
| Components | (wt %) |
| Icariside II | 5.00 |
| Polyvinyl alcohol | 13.00 |
| L-ascorbic acid-2-magnesium | 1.00 |
| phosphate | |
| Lauroyl hydroxyproline | 1.00 |
| Water-soluble collagen | 2.00 |
| (1% aqueous solution) | |
| 1,3-butylene glycol | 3.00 |
| Ethanol | 5.00 |

Purified water was added to the above components to make a total of 100 wt %, and a cosmetic pack having said composition was prepared.

Formulation Example 5: Preparation of essence

| | Content |
|---|---|
| Components | (wt %) |
| Icariside II | 2.00 |
| Hydroxyethylene cellulose | 12.00 |
| (2% aqueous solution) | |
| Xanthan gum | 2.00 |
| (2% aqueous solution) | |
| 1,3-butylene glycol | 6.00 |
| Concentrated glycerin | 4.00 |
| Sodium hyaluronate | 5.00 |
| (1% aqueous solution) | |

Purified water was added to the above components to make a total of 100 wt %, and essence having said composition was prepared.

Formulation Example 6: Preparation of powders

| Components | Content (mg) |
|---|---|
| Icariside II | 300 |
| Lactose | 100 |
| Talc | 10 |

Said components were mixed with each other and packed in airtight cloth, thus preparing powders.

Formulation Example 7: Preparation of tablets

| Components | Content (mg) |
|---|---|
| Icariside II | 50 |
| Corn starch | 100 |
| Lactose | 100 |
| Magnesium stearate | 2 |

The above components were mixed with each other, and then tableted according to a conventional method, thus preparing tablets.

Formulation Example 8: Preparation of capsules

| Components | Content (mg) |
|---|---|
| Icariside II | 50 |
| Corn starch | 100 |
| Lactose | 100 |
| Magnesium stearate | 2 |

According to a conventional method for preparing capsules, the above components were mixed with each other and filled in gelatin capsules, thus preparing capsules.

Formulation Example 9: Preparation of injection solution

| Components | Content (mg) |
|---|---|
| Icariside II | 50 |
| Sterilized distilled water | qs |
| for injection | |
| pH adjuster | qs |

According to a method for preparing an injection solution, a 2-ml ampoule containing the above components and contents was prepared.

Formulation Example 10: Preparation of liquid formulation

| Components | Contents | |
|---|---|---|
| Icariside II | 100 | mg |
| Isomerized sugar | 10 | g |
| Mannitol | 5 | g |

| | |
|---|---|
| Purified water | qs |

According to a conventional method for preparing a liquid formulation, each of the above components was dissolved in purified water, and a suitable amount of lemon flavor was added thereto. Then, the above components were mixed with each other, and purified water was added thereto to make a total of 100 ml. Then, the solution was filled in a brown bottle and sterilized, thus preparing a liquid formulation.

### INDUSTRIAL APPLICABILITY

As described above, the composition containing icariside II can inhibit the activity of alpha-glucosidase to interfere with the normal glycosylation of tyrosinase, thus improving pigmentation. Accordingly, the composition will be useful as a skin whitening cosmetic composition or pharmaceutical composition.

1. A method for preparing icariside II represented by Formula 1, the method comprising extracting icariside II from an Epimedium plant using an organic solvent or a mixture of the organic solvent with water: 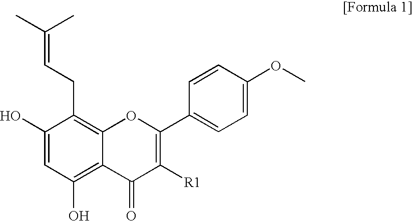
wherein R1 is rhamnopyranose.

2. The method of claim 1, wherein the organic solvent is selected from the group consisting of ethanol, methanol, butanol, ether, ethyl acetate and chloroform.

3. The method of claim 1, wherein Epimedium plant is selected from the group consisting of Epimedium brevicornum Maxim., Epimedium grandiflorum Morr., Epimedium koreanum Nakai, Epimedium pubescens Maxim., Epimedium sagittatum Maxim. and Epimedium wushanense.

4. A method for preparing icariside II represented by Formula 1, the method comprising selectively removing a glucose moiety from icariin using an enzyme or a microorganism producing the enzyme without degrading rhamnose: 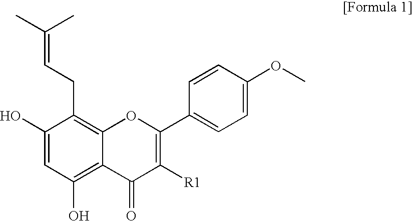
wherein R1 is rhamnopyranose.

5. The method of claim 4, wherein the enzyme is one or more selected from the group consisting of glucosidase, arabinosidase, xylosidase, cellulase, glucuronidase, galactosidase and amyloglucosidase.

6. The method of claim 4, wherein the microorganism is one or more selected from the group consisting of Aspergillus sp., Bacillus sp., Penicillium sp., Rhizopus sp., Rhizomucor sp., Talaromyces sp., Bifidobacterium sp., Mortierella sp., Cryptococcus sp., and Microbacterium sp.

7. A cosmetic composition containing, as an active ingredient, icariside II represented by Formula 1: 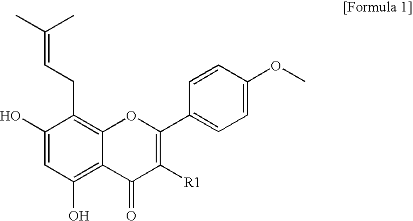
wherein R1 is rhamnopyranose.

8. Use of a cosmetic composition, containing, as an active ingredient, icariside II represented by Formula 1, for skin whitening: 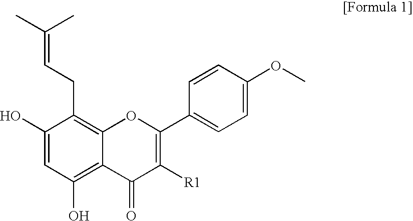
wherein R1 is rhamnopyranose.
